(19) United States
(12) Patent Application Publication
(10) Pub. No.: US 2021/0208142 A1
(43) Pub. Date: Jul. 8, 2021

(54) METHOD FOR DETECTION AND ANALYSIS OF CEREBROSPINAL FLUID ASSOCIATED UBE3A

(71) Applicant: University of South Florida, Tampa, FL (US)

(72) Inventors: Edwin John Weeber, South Plainfield, NJ (US); Hayden Elyse Greene, St. Petersburg, FL (US)

(21) Appl. No.: 17/180,189

(22) Filed: Feb. 19, 2021

Related U.S. Application Data
(63) Continuation-in-part of application No. PCT/US2020/015380, filed on Jan. 28, 2020.
(60) Provisional application No. 62/798,712, filed on Jan. 30, 2019.

(51) Int. Cl. G01N 33/573

(52) U.S. Cl. CPC G01N 33/573; G01N 2333/9015; A61K 48/00; G01N 2800/2814; G01N 2800/52

(57) ABSTRACT

A method and kit for diagnosing Angelman Syndrome by detecting and analyzing ubiquitination of the UBE3A protein in cerebrospinal fluid is presented. CSF is collected from a patient and incubated with a substrate, such as S5a, and ubiquitin. Ubiquitination of the substrate by UBE3A is measured and compared to a control sample for biochemical diagnosis of Angelman Syndrome. A method of determining the efficacy of a treatment is also presented in which CSF is collected from the patient both prior to and after treatment and incubated with a substrate and ubiquitin. Ubiquitination of the substrate by UBE3A is measured and an increase in ubiquitination in the sample obtained after treatment as compared to the reference sample collected prior to treatment indicates efficacy of the treatment.

### CROSS REFERENCE TO RELATED APPLICATIONS

This application is a continuation in part of and claims priority to International Application No. PCT/US2020/015380, entitled “Method for Detection and Analysis of Cerebrospinal Fluid Associated UBE3A”, filed Jan. 28, 2020, which is a nonprovisional of and claims priority to U.S. Provisional Application No. 62/798,712 entitled “Method for Detection and Analysis of Cerebral Spinal Fluid Associated UBE3A”, filed Jan. 30, 2019, the contents of each of which are hereby incorporated by reference into this disclosure.

### FIELD OF INVENTION

This invention relates to diagnostics for Angelman syndrome. Specifically, the invention provides a method of detecting and analyzing the UBE3A enzyme in cerebrospinal fluid.

### BACKGROUND OF THE INVENTION

Angelman syndrome (AS) is a genetic disorder affecting neurons, estimated to affect about one in every 15,000 births (Clayton-Smith, Clinical research on Angelman syndrome in the United Kingdom: observations on 82 affected individuals. Am J Med Genet. 1993 Apr. 1; 46(1):12-5), though the actual number of diagnosed AS cases is greater likely due to misdiagnosis.

Angelman syndrome is a continuum of impairment, which presents with delayed and reduced intellectual and developmental advancement, most notably regarding language and motor skills. In particular, AS is defined by little or no verbal communication, with some non-verbal communication, ataxia, and disposition that includes frequent laughing and smiling and excitable movement.

More advanced cases result in severe mental retardation, seizures that may be difficult to control that typically begin before or by three years of age, frequent laughter (Nicholls, New insights reveal complex mechanisms involved in genomic imprinting. Am J Hum Genet. 1994 May; 54(5):733-40), miroencephaly, and abnormal EEG. In severe cases, patients may not develop language or may only have use of 5-10 words. Movement is commonly jerky and walking commonly is associated with hand flapping and a stiff-gait. The patients are commonly epileptic, especially earlier in life, and suffer from sleep apnea, commonly only sleeping for 5 hours at a time. They are social and desire human contact. In some cases, skin and eyes may have little or no pigment, they may possess sucking and swallowing problems, sensitivity to heat, and a fixation to water bodies. Studies in UBE3A-deficient mice show disturbances in long-term synaptic plasticity. There are currently no cures for Angelman syndrome, and treatment is palliative. For example, anticonvulsant medication is used to reduce epileptic seizures, and speech and physical therapy are used to improve language and motor skills.

The gene UBE3A is responsible for AS and it is unique in that it is one of a small family of human imprinted genes. UBE3A, found on chromosome 15, encodes for the homologous to E6AP C terminus (HECT) protein (E6-associated protein (E6AP) (Kishino, et al., UBE3A/E6-AP mutations cause Angelman syndrome. Nat Gen. 1997 Jan. 15.15(1):70-3). UBE3A undergoes spatially-defined maternal imprinting in the brain; thus, the paternal copy is silenced via DNA methylation (Albrecht, et al., Imprinted expression of the murine Angelman syndrome gene, Ube3a, in hippocampal and Purkinje neurons. Nat Genet. 1997 September; 17(1):75-8). As such, only the maternal copy is active, the paternal chromosome having little or no effect on the proteosome of the neurons in that region of the brain. Inactivation, translocation, or deletion of portions of chromosome 15 therefore results in uncompensated loss of function. Some studies suggest improper E3-AP protein levels alter neurite contact in Angelman syndrome patients (Tonazzini, et al., Impaired neurite contract guidance in ubiquitin ligase E3a (Ube3a)-deficient hippocampal neurons on nanostructured substrates. Adv Healthc Mater. 2016 April; 5(7):850-62).

The majority of Angelman's syndrome cases (70%) occur through a de novo deletion of around 4 Mb from 15q11-q13 of the maternal chromosome which incorporates the UBE3A gene (Kaplan, et al., Clinical heterogeneity associated with deletions in the long arm of chromosome 15: report of 3 new cases and their possible significance. Am J Med Genet. 1987 September; 28(1):45-53), but it can also occur as a result of abnormal methylation of the maternal copy, preventing its expression (Buiting, et al., Inherited microdeletions in the Angelman and Prader-Willi syndromes define an imprinting centre on human chromosome 15. Nat Genet. 1995 April; 9(4):395-400; Gabriel, et al., A transgene insertion creating a heritable chromosome deletion mouse model of Prader-Willi and Angelman syndrome. Proc Natl Acad Sci U.S.A. 1999 August; 96(16):9258-63) or uniparental disomy in which two copies of the paternal gene are inherited (Knoll, et al., Angelman and Prader-Willi syndromes share a common chromosome 15 deletion but differ in parental origin of the deletion. Am J Med Genet. 1989 Fed; 32(2):285-90; Malcolm, et al., Uniparental paternal disomy in Angelman's syndrome. Lancet. 1991 Mar. 23; 337(8743):694-7). The remaining AS cases arise through various UBE3A mutations of the maternal chromosome or they are diagnosed without a genetic cause (12-15UBE3A codes for the E6-associated protein (E6-AP) ubiquitin ligase. E6-AP is an E3 ubiquitin ligase, therefore it exhibits specificity for its protein targets, which include the tumor suppressor molecule p53 (Huibregtse, et al., A cellular protein mediates association of p53 with the E6 oncoprotein of human papillomavirus types 16 or18. EMBO J. 1991 December; 10(13):4129-35), a human homologue to the yeast DNA repair protein Rad23 (Kumar, et al., Identification of HHR23A as a substrate for E6-associated protein-mediated ubiquitination. J Biol Chem. 1999 Jun. 25; 274(26):18785-92), E6-AP itself, and Arc, the most recently identified target (Nuber, et al., The ubiquitin-protein ligase E6-associated protein (E6-AP) serves as its own substrate. Eur J Biochem. 1998 Jun. 15; 254(3):643-9; Greer, et al., The Angelman Syndrome protein Ube3A regulates synapse Development by ubiquitinating arc. Cell. 2010 Mar. 5; 140(5): 704-16).

Mild cases are likely due to a mutation in the UBE3A gene at chromosome 15q11-13, which encodes for E6-AP ubiquitin ligase protein of the ubiquitin pathway, and more severe cases resulting from larger deletions of chromosome 15. Commonly, the loss of the UBE3A gene in the hippocampus and cerebellum result in Angelman syndrome, though single loss-of-function mutations can also result in the disorder.

The anatomy of the mouse and human AS brain shows no major alterations compared to the normal brain, indicating the cognitive deficits may be biochemical in nature as opposed to developmental (Jiang, et al., Mutation of the Angelman ubiquitin ligase in mice causes increased cytoplasmic p53 and deficits of contextual learning and long-term potentiation. Neuron. 1998 October; 21(4):799-811; Davies, et al., Imprinted gene expression in the brain. Neurosci Biobehav Rev. 2005 May; 29(3):421-430). An Angelman syndrome mouse model possessing a disruption of the maternal UBE3A gene through a null mutation of exon 2 (Jiang, et al., Mutation of the Angelman ubiquitin ligase in mice causes increased cytoplasmic p53 and deficits of contextual learning and long-term potentiation. Neuron. 1998 October; 21(4):799-811) was used. This model has been incredibly beneficial to the field of AS research due to its ability in recapitulating the major phenotypes characteristic of AS patients. For example, the AS mouse has inducible seizures, poor motor coordination, hippocampal-dependent learning deficits, and defects in hippocampal LTP. Cognitive deficits in the AS mouse model were previously shown to be associated with abnormalities in the phosphorylation state of calcium/calmodulin-dependent protein kinase II (CaMKII) (Weeber, et al., Derangements of hippocampal calcium/calmodulin-dependent protein kinase II in a mouse model for Angelman mental retardation syndrome. J Neurosci. 2003 April; 23(7):2634-44). There was a significant increase in phosphorylation at both the activating Thr²⁸⁶ site as well as the inhibitory Thr³⁰⁵ site of αCaMKII without any changes in total enzyme level, resulting in an overall decrease in its activity. There was also a reduction in the total amount of CaMKII at the postsynaptic density, indicating a reduction in the amount of active CaMKII. Crossing a mutant mouse model having a point mutation at the Thr³⁰⁵ site preventing phosphorylation with the AS mouse rescued the AS phenotype. i.e. seizure activity, motor coordination, hippocampal-dependent learning, and LTP were restored similar to wildtype levels. Thus, postnatal expression of αCaMKII suggests that the major phenotypes of the AS mouse model are due to postnatal biochemical alterations as opposed to a global developmental defect (Bayer, et al., Developmental expression of the CaM kinase II isoforms: ubiquitous γ- and δ-CaM kinase II are the early isoforms and most abundant in the developing nervous system. Brain Res Mol Brain Res. 1999 Jun. 18; 70(1):147-54).

Current methods to diagnose Angelman syndrome consist mainly of genetic analysis given that bi-allelic expression elsewhere in the body makes diagnosis by blood or any peripheral tissue impossible. Alterations in promoted regions or paternal UBE3A disomy can make diagnosis difficult using genetic analysis. Furthermore, between about 5-7% of Angelman syndrome cases have no molecular diagnosis, only a clinical diagnosis without a biochemical confirmation. Accordingly, what is needed is a method of diagnosing Angelman syndrome that allows for a biochemical confirmation.

### SUMMARY OF INVENTION

Angelman syndrome (AS) is a difficult to diagnose, rare disorder associated with the absence of the UBE3A protein in the central nervous system. As noted above, bi-allelic expression elsewhere in the body makes diagnosis by blood or any peripheral tissue impossible. Genetic analysis is the current method of diagnosis, however alterations in promoted regions or paternal UBE3A disomy can make diagnosis difficult. The inventors have addressed this problem by developing a method allowing for biochemical confirmation through the detection and analyzation of UBE3A in cerebrospinal fluid. The method qualitatively detects and quantitatively analyzes the UBE3A enzyme in cerebrospinal fluid and thus may be used as a diagnostic tool to detect the absence of UBE3A in the CSF in individuals that do not show a typical disruption (deletion/mutation) to the maternal UBE3A allele.

In addition to clinical diagnosis with biochemical confirmation, the present method can be used in biomarker detection to determine the effectiveness of a given treatment in an individual through the analysis of about 15-20 ul of cerebrospinal fluid (CSF). Multiple strategies are now being developed to treat AS. These include the activation of the paternal allele, AAV-mediated gene therapy and protein replacement therapy. Determination of whether these treatments are having the desired effect in a clinical trial is impossible without the ability to detect neuronal derived UBE3A.

In an embodiment, a method of diagnosing and treating a neurodegenerative disease characterized by UBE3A deficiency in a patient is presented comprising: extracting a sample of cerebrospinal fluid from the patient; combining and incubating the sample of cerebrospinal fluid with a substrate protein and ubiquitin in a reaction tube; terminating reaction of the contents at set time points; quantifying UBE3A enzymatic activity in the patient sample; comparing the UBE3A enzymatic activity of the patient sample to a control sample; and administering treatment for the neurodegenerative disease if an absence or decrease of UBE3A enzymatic activity in the patient sample as compared to the control sample is found.

The neurodegenerative disease may be selected from the group consisting of Angelman's Syndrome, Huntington's disease, Alzheimer's disease, Parkinson's disease, amyotrophic lateral sclerosis, autistic spectrum disorders, epilepsy, multiple sclerosis, Prader-Willi syndrome, Fragile X syndrome, Rett syndrome and Pick's Disease. In a preferred embodiment, the neurodegenerative disease may be Angelman syndrome.

The substrate protein may be selected from the group consisting of S5a, Sox9, Rad23, p53, MCMI, p2′7, promyelocytic leukemia tumor suppressor (PML), amplified in breast cancer 1 (AIB1), HHR23A, a-Synuclein, C/EBPa and UBE3A. In a preferred embodiment, the substrate is S5a.

The UBE3A enzymatic activity may be quantified by measuring the amount of ubiquitination of the substrate protein by Western blot.

The treatment may be selected from the group consisting of activation of the paternal allele, AAV-mediated gene therapy and protein replacement therapy.

In another embodiment, a method of determining efficacy of treatment of a neurodegenerative disease characterized by UBE3A deficiency in a patient is presented comprising: collecting a reference sample of cerebrospinal fluid from the patient prior to administering treatment; administering a treatment to the patient having Angelman Syndrome; collecting at least one sample of cerebrospinal fluid from the patient at least one time period after treatment; incubating the reference sample with a substrate protein and ubiquitin in a first reaction tube; incubating the at least one sample collected at the at least one time period after the administration of the treatment with the substrate protein and the ubiquitin in a second reaction tube; terminating reaction of the contents of each of the reaction tubes at set time points; quantifying UBE3A enzymatic activity in both the reference sample and the at least one sample collected at the at least one time period after the administration of the treatment; and comparing the UBE3A enzymatic activity in the at least one sample collected at the at least one time period after the administration of the treatment to the reference sample. An increase in the amount of UBE3A enzymatic activity in the at least one sample collected at the at least one time period after the administration of the treatment as compared to the reference sample indicates efficacious treatment.

The neurodegenerative disease may be selected from the group consisting of Angelman's Syndrome, Huntington's disease, Alzheimer's disease, Parkinson's disease, amyotrophic lateral sclerosis, autistic spectrum disorders, epilepsy, multiple sclerosis, Prader-Willi syndrome, Fragile X syndrome, Rett syndrome and Pick's Disease. In a preferred embodiment, the neurodegenerative disease may be Angelman syndrome.

The substrate protein may be selected from the group consisting of S5a, Sox9, Rad23, p53, MCM7, p2′7, promyelocytic leukemia tumor suppressor (PML), amplified in breast cancer 1 (AIB1), HHR23A, a-Synuclein, C/EBPa and UBE3A. In a preferred embodiment, the substrate is S5a.

The UBE3A enzymatic activity may be quantified by measuring the amount of ubiquitination of the substrate protein by Western blot.

The treatment may be selected from the group consisting of activation of the paternal allele, AAV-mediated gene therapy and protein replacement therapy.

In an embodiment, a kit for diagnosing Angelman's Syndrome is presented comprising: a substrate protein wherein the substrate protein is selected from the group consisting of S5a, Sox9, Rad23, p53, MCM7, p2′7, promyelocytic leukemia tumor suppressor (PML), amplified in breast cancer 1 (AIB1), HHR23A, a-Synuclein, C/EBPa and UBE3A; an enzyme solution; an adenosine triphosphate (ATP) solution; a ubiquitin solution; a redox reagent such as dithiothreitol (DTT); and printed instructions for use of the kit in diagnosing Angelman's syndrome using cerebrospinal fluid from a subject. All components of the kit are contained in separate containers.

### BRIEF DESCRIPTION OF THE DRAWINGS

For a fuller understanding of the invention, reference should be made to the following detailed description, taken in connection with the accompanying drawings, in which:

FIG. 1A-C are a series of images depicting UBE3A is located in the cerebral spinal fluid. (A & B) Analysis of human and rat tissue and CSF showing the lack of the maternal UBE3A allele results in a marked reduction of protein; (C) Western blot showing AS humans have a significant deficit in UBE3A compared to typical humans. Hippocampal tissue, order left to right: Recombinant E6AP (human isoform 2), Lanes 2-4 Rat wild-type, AS and knock-out, Lane 5 human neuro-typical, Lane 6 human AS;

FIG. 1D is a Western blot showing AS rats have a significant deficit in UBE3A compared to WT rats.

FIG. 2 is a series of images depicting AAV-UBE3A mediated expression in the AS rat shows increased UBE3A in the CSF 5 weeks after viral transfection. This indicates that UBE3A in the CSF is derived from neuronal expression and that exogenous gene therapy is sufficient to detect UBE3A in the CSF.

FIG. 3 is a series of images depicting the UBE3A assay using S5a as a substrate for UBE3A. CSF from a rat was incubated with recombinant S5a and stopped at specific time points. Analysis of the reduction of the 50 kDa S5a protein as it is ubiquinated changes its molecular weight. DUB is 9-hour assay incubated with deubiquinating enzymes showing the increase in molecular weight is due to ubiquination.

FIG. 4 is a graph depicting CSF UBE3A assay from CSF obtained from wild-type, UBE3A maternal deficient Rats (AS) and UBE3A knock out rats. Reduction over time of S5a substrate reveals that other ubiquitin ligases are present in Rat CSF that are not UBE3A, but the differential activity in the AS rat is due to UBE3A exclusively.

FIG. 5A-B are a series of Western blots illustrating UBE3A maintains its catalytic activity within the extracellular space. (A) Representative image of WT diasylate UBE3A and S5a assay Western blots at 9 different time points (min); (B) Representative Western blot of WT diasylate with UBE3A immunoprecipitated, AS diasylate, WT CSF, UBE3A and S5a assay Western blots at 3 different time points (min).

FIG. 6A is an image depicting extracellular UBE3A undergoes activity dependent regulation following fear conditioning. (A) Representative images of Western blots.

FIG. 6B is an image depicting extracellular UBE3A undergoes activity dependent regulation following fear conditioning. (B) Densitometry of Western blot (Baseline, n=8)

FIG. 6C is an image depicting extracellular UBE3A undergoes activity dependent regulation following fear conditioning. (C) Densitometry of Western blot (Shock, n=8)

FIG. 6D is an image depicting extracellular UBE3A undergoes activity dependent regulation following fear conditioning. (D) Densitometry of Western blots (No shock, n=9).

FIG. 7 is a series of images depicting Human CSF from typical or AS source showing S5a ubiquination over time. Human typical CSF has significantly more ability to ubiquinate S5a compared to CSF from AS.

### DETAILED DESCRIPTION OF THE PREFERRED EMBODIMENT

In the following detailed description of the preferred embodiments, reference is made to the accompanying drawings, which form a part hereof, and within which are shown by way of illustration specific embodiments by which the invention may be practiced. It is to be understood that other embodiments may be utilized, and structural changes may be made without departing from the scope of the invention.

### Definitions

Unless otherwise defined, all technical and scientific terms used herein have the same meaning as commonly understood by one of ordinary skill in the art to which this invention belongs. Although any methods and materials similar or equivalent to those described herein can be used in the practice or testing of the present invention, some potential and preferred methods and materials are described herein. All publications mentioned herein are incorporated herein by reference in their entirety to disclose and describe the methods and/or materials in connection with which the publications are cited. It is understood that the present disclosure supercedes any disclosure of an incorporated publication to the extent there is a contradiction.

As used herein, the singular forms “a,” “an” and “the” include plural referents unless the context clearly dictates otherwise.

All numerical designations, such as pH, temperature, time, concentration, and molecular weight, including ranges, are approximations which are varied up or down by increments of 1.0 or 0.1, as appropriate. It is to be understood, even if it is not always explicitly stated that all numerical designations are preceded by the term “about”. It is also to be understood, even if it is not always explicitly stated, that the reagents described herein are merely exemplary and that equivalents of such are known in the art and can be substituted for the reagents explicitly stated herein.

As used herein, the term “comprising” is intended to mean that the products, compositions, and methods include the referenced components or steps, but not excluding others. “Consisting essentially of” when used to define products, compositions, and methods, shall mean excluding other components or steps of any essential significance. Thus, a composition consisting essentially of the recited components would not exclude trace contaminants and pharmaceutically acceptable carriers. “Consisting of” shall mean excluding more than trace elements of other components or steps.

As used herein, “about” means approximately or nearly and in the context of a numerical value or range set forth means±15% of the numerical.

As used herein “patient” is used to describe an animal, preferably a human, to whom treatment is administered, including prophylactic treatment with the compositions of the present invention. “Subject” and “patient” are used interchangeably herein.

As used herein “animal” means a multicellular, eukaryotic organism classified in the kingdom Animalia or Metazoa. The term includes, but is not limited to, mammals. Non-limiting examples include rodents, mammals, aquatic mammals, domestic animals such as dogs and cats, farm animals such as sheep, pigs, cows and horses, and humans. Wherein the terms “animal” or the plural “animals” are used, it is contemplated that it also applies to any animals.

“Neurodegenerative disorder” or “neurodegenerative disease” as used herein refers to any abnormal physical or mental behavior or experience where the death or dysfunction of neuronal cells is involved in the etiology of the disorder. Further, the term “neurodegenerative disease” as used herein describes “neurodegenerative diseases” which are associated with UBE3A deficiencies. Exemplary neurodegenerative diseases include Angelman's Syndrome, Huntington's disease, Alzheimer's disease, Parkinson's disease, amyotrophic lateral sclerosis, autistic spectrum disorders, epilepsy, multiple sclerosis, Prader-Willi syndrome, Fragile X syndrome, Rett syndrome and Pick's Disease.

The term “biomarker” is used herein to refer to a molecule whose level of nucleic acid or protein product has a quantitatively differential concentration or level with respect to an aspect of a biological state of a subject. “Biomarker” is used interchangeably with “marker” herein. The level of the biomarker can be measured at both the nucleic acid level as well as the polypeptide level. At the nucleic acid level, a nucleic acid gene or a transcript which is transcribed from any part of the subject's chromosomal and extrachromosomal genome, including for example the mitochondrial genome, may be measured. Preferably an RNA transcript, more preferably an RNA transcript includes a primary transcript, a spliced transcript, an alternatively spliced transcript, or an mRNA of the biomarker is measured. At the polypeptide level, a pre-propeptide, a propeptide, a mature peptide or a secreted peptide of the biomarker may be measured. A biomarker can be used either solely or in conjunction with one or more other identified biomarkers so as to allow correlation to the biological state of interest as defined herein. Biomarkers of the present invention include UBE3A.

The term “peptide” as used herein refers to short polymers formed from the linking, in a defined order, of α-amino acids. The link between one amino acid residue and the next is known as an amide bond or a peptide bond. Proteins are polypeptide molecules (or consist of multiple polypeptide subunits). The distinction is that peptides are short, and polypeptides/proteins are long. There are several different conventions to determine these. Peptide chains that are short enough to be made synthetically from the constituent amino acids are called peptides, rather than proteins, with one commonly understood dividing line at about 50 amino acids in length.

The term “polypeptide” as used herein refers to a compound made up of a single-chain of amino acid residues that are linked by peptide bonds. The term “protein” may be synonymous with the term “polypeptide” or may refer, in addition, to a complex of two or more polypeptides. Generally, polypeptides and proteins are formed predominantly of naturally occurring amino acids.

The term “expression level” as used herein refers to detecting the amount or level of expression of a biomarker of the present invention. The act of actually detecting the expression level of a biomarker refers to the act of actively determining whether a biomarker is expressed in a sample or not. This act can include determining whether the biomarker expression is upregulated, downregulated or substantially unchanged as compared to a control level expressed in a sample. The expression level in some cases may refer to detecting transcription of the gene encoding a biomarker protein and/or to detecting translation of the biomarker protein.

The term “quantifying” or “quantitating” when used in the context of quantifying transcription levels of a gene can refer to absolute or relative quantification. Absolute quantification can be achieved by including known concentration(s) of one or more target nucleic acids and referencing the hybridization intensity of unknowns with the known target nucleic acids (e.g. through the generation of a standard curve). Alternatively, relative quantification can be achieved by comparison of hybridization signals between two or more genes, or between two or more treatments to quantify the changes in hybridization intensity and, by implication transcription level.

Methods to measure protein/polypeptide expression levels of selected biomarkers in the present invention include, but are not limited to: Western blot, immunoblot, enzyme-linked immunosorbent as say (ELISA), radioimmunoassay (RIA), immunoprecipitation, surface plasmon resonance, chemiluminescence, fluorescent polarization, phosphorescence, immunohistochemical analysis, liquid chromatography mass spectrometry (LC-MS), matrix-assisted laser desorption/ionization time-of-flight (MALDI-TOF), mass spectrometry, microcytometry, microarray, microscopy, fluorescence activated cell sorting (FACS), flow cytometry, and assays based on a property of the protein including but not limited to DNA binding, ligand binding, or interaction with other protein partners.

The term “sample,” as used herein, refers to a composition that is obtained or derived from a subject and/or individual of interest that contains a cellular and/or other molecular entity that is to be characterized and/or identified, for example, based on physical, biochemical, chemical, and/or physiological characteristics. For example, the phrase “disease sample” and variations thereof refers to any sample obtained from a subject of interest that would be expected or is known to contain the cellular and/or molecular entity that is to be characterized. In some embodiments, the term “sample” refers to an amount of cerebrospinal fluid (CSF).

The term “control sample” as used herein refers to a sample, standard or level that is used for comparison purposes. In some embodiments, the control sample may be obtained from a healthy and/or non-diseased part of the body of an individual who is not the subject. For example, the control sample may be a sample of CSF obtained from a healthy individual who does not have Angelman syndrome. This healthy individual may be referred to as “normal” as defined herein. In other embodiments which determine the efficacy of a given treatment, the control sample may be obtained from an untreated part of the body of the subject. For example, the control sample to determine efficacy of a given treatment may be a sample of CSF taken from the subject prior to any treatment being administered. This control sample is then compared to a sample taken after treatment has been administered to determine the efficacy of the treatment. The terms “control sample” and “reference sample” are used interchangeably herein.

“UBE3A deficiency” as used herein refers to the amount of UBE3A present in a patient being less than the amount of UBE3A present in a normal sample. In some embodiments, the UBE3A deficiency may be due to a mutation or deletion in the UBE3A gene.

The term “normal” as used herein refers to a sample or patient which are assessed as not having Angelman syndrome or any other neurodegenerative disease or any other UBE3A deficient neurological disorder.

The terms “diagnosing” or “diagnosis” as used herein refers to identification or classification of a molecular or pathological state, disease, or condition (e.g., a neurodegenerative disorder). As used herein, “diagnosing” refers to the identification of a neurodegenerative disease, particularly identification of a UBE3A-deficient disease such as Angelman syndrome.

In some embodiments, the method described herein may use the detection of UBE3A as a biomarker for evaluation of the efficacy of a given treatment for a disease in a patient.

The evaluation of the efficacy of the treatment for a disease can be assessed by comparing the level of the biomarker UBE3A in CSF at a first timepoint before administration of the treatment to the level of the biomarker at a second timepoint which occurs at a specified interval after the administration of the treatment. An increase in the level of UBE3A at the second timepoint after administration of the treatment as compared to the level measured at the first timepoint is indicative of efficacious treatment. A level of UBE3A at the second timepoint that is equal to or less than the amount measured at the first timepoint is indicative of the treatment being ineffective. In some embodiments, the level of UBE3A is measured according to its ubiquitination to a substrate such as S5a. The ubiquitination can be measured over time in some embodiments.

“Treatment” or “treating” as used herein refers to any of: the alleviation, amelioration, elimination and/or stabilization of a symptom, as well as delay in progression of a symptom of a particular disorder. For example, “treatment” of a neurodegenerative disease may include any one or more of the following: amelioration and/or elimination of one or more symptoms associated with the neurodegenerative disease, reduction of one or more symptoms of the neurodegenerative disease, stabilization of symptoms of the neurodegenerative disease, and delay in progression of one or more symptoms of the neurodegenerative disease.

“Administration” or “administering” is used to describe the process in which therapeutics used to treat neurodegenerative diseases such as Angelman syndrome, alone or in combination with other therapeutics, are delivered to a patient. The composition may be administered in various ways including injection into the central nervous system including the brain, including but not limited to, intrastriatal, intrahippocampal, ventral tegmental area (VTA) injection, intracerebral, intracerebellar, intramedullary, intranigral, intraventricular, intracisternal, intracranial, intraparenchymal including spinal cord and brain stem; oral; parenteral (referring to intravenous and intraarterial and other appropriate parenteral routes); intrathecal; intramuscular; subcutaneous; rectal; and nasal, among others. Each of these conditions may be readily treated using other administration routes of compounds of the present invention to treat a disease or condition.

The dosing of compounds and compositions to obtain a therapeutic or prophylactic effect is determined by the circumstances of the patient, as known in the art. The dosing of a patient herein may be accomplished through individual or unit doses of the compounds or compositions herein or by a combined or prepackaged or pre-formulated dose of a compounds or compositions. An average 40 g mouse has a brain weighing 0.416 g, a 160 g mouse has a brain weighing 1.02 g, and a 250 g mouse has a brain weighing 1.802 g. An average 400 g rat has a brain weighing 2 g. An average human brain weighs 1508 g, which can be used to direct the amount of therapeutic needed or useful to accomplish the treatment described herein.

As used herein, the phrase “pharmaceutically acceptable carrier” means any of the standard pharmaceutically acceptable carriers. The pharmaceutically acceptable carrier can include diluents, adjuvants, and vehicles, as well as implant carriers, and inert, non-toxic solid or liquid fillers, diluents, or encapsulating material that does not react with the active ingredients of the invention. Examples include, but are not limited to, phosphate buffered saline, physiological saline, water, and emulsions, such as oil/water emulsions. The carrier can be a solvent or dispersing medium containing, for example, ethanol, polyol (for example, glycerol, propylene glycol, liquid polyethylene glycol, and the like), suitable mixtures thereof, and vegetable oils. Formulations are described in a number of sources that are well known and readily available to those skilled in the art. For example, Remington's Pharmaceutical Sciences (Martin E W [1995] Easton Pa., Mack Publishing Company, 19^{th} ed.) describes formulations which can be used in connection with the subject invention.

As used herein, the term “therapeutically effective amount” refers to that amount of a therapy (e.g., a therapeutic agent or vector) sufficient to result in the amelioration of Angelman syndrome or other UBE3A-related disorder or one or more symptoms thereof, prevent advancement of Angelman syndrome or other UBE3A-related disorder, or cause regression of Angelman syndrome or other UBE3A-related disorder.

Angelman syndrome (AS) is a rare disorder resulting from the disruption of the maternal UBE3A gene. The UBE3A gene is imprinted in the central nervous system (CNS) meaning that only the maternal gene expresses UBE3A protein, while the paternal UBE3A gene is silenced. The method described herein qualitatively detects and quantitatively analyzes the UBE3A enzyme in cerebrospinal fluid and thus may be used as a diagnostic tool to detect the absence of UBE3A in the CSF in individuals that do not show a typical disruption (deletion/mutation) to the maternal UBE3A allele.

The method may also be used as a biomarker to assess effectiveness of any known treatment or even of proposed, upcoming clinical trials in AS focused on gene replacement, protein replacement or re-activation and unsilencing of the paternal UBE3A allele. The examples provided herein illustrate the various embodiments of the invention.

Methods

Immunological Analysis

For CSF Westerns in both rat and human samples, all CSF samples were concentrated using Amicon Ultra 0.5 mL centrifugal filters per the manufacturer instructions (100 kDa, Sigma Aldrich). Concentrated CSF was boiled at 95° C. for 5 minutes in 4× Laemlli loading buffer (Bio-Rad) prior to gel electrophoresis. To assess UBE3A protein levels, 22.5 μl of concentrated CSF was separated by 4-15% SDS-polyacrylamide gel electrophoresis (Criterion™ TGX™ Precast Midi Protein Gel, Bio-Rad) and transferred to a PVDF membrane (Invitrogen). The membrane was blocked for 1 hour at room temperature in 5% non-fat milk in TBST, followed by an overnight 4° C. incubation with primary antibody (mouse monoclonal anti-E6AP antibody, 1:1000, Sigma-Aldrich). The next day, the membrane was rinsed with three 10-minute washes of TBS-T and incubated at room temperature for 1 hour with horseradish peroxidase-conjugated secondary antibody in 5% non-fat milk in TBS-T (goat anti-mouse IgG, 1:2000, Bethyl Laboratories). Following secondary incubation, the membrane was rinsed 4-5 times with TBS-T for 1 hour. The Enhanced Chemiluminescence Detection System (Pierce Chemical Company) was used to visualize the immunostaining using ChemiDoc™ XRS+ System with Image Lab™ Software (Bio-Rad).

Tissue and CSF SSA Activity Assay

Tissue and CSF S5A Activity Assay was performed using a E6AP Ubiquitin Ligase Kit for the S5a substrate (Boston Biochem).

In each reaction tube, 18 μl of CSF was added to 3 μl 10×E2 enzyme, Mg²⁺-ATP solution, and His6-55a substrate protein provided by the kit. To initiate the reaction, 30 of 10× ubiquitin was added to the reaction tube. The reaction tubes were then placed in an incubator at 37° C. for the entirety of the assay. At each time point collection, 30 from each reaction tube were added to a tube with 5× loading buffer and 1 ul of 1M DTT, terminating the reaction. Samples were then flash frozen on dry ice and stored at −80° C. until Western blot analysis.

For the Western blots, 9 ul of each time point were loaded into a 10 well, 1.0 mm thickness, 10% SDS-polyacrylamide gel and subjected to electrophoresis. Proteins were transferred to a PVDF membrane (Invitrogen). The membrane was blocked for 1 hour at room temperature in 5% non-fat milk in TBST, followed by an overnight 4° C. incubation with anti-S5a primary antibody provided by the kit (goat polyclonal, 1:1000, Boston Biochem). The next day, the membrane was rinsed with three 10-minute washes of TBS-T and incubated at room temperature for 1 hour with horseradish peroxidase-conjugated secondary antibody in 5% non-fat milk in TBS-T (donkey anti-goat IgG, 1:5000, EMD Millipore). Following secondary incubation, the membrane was rinsed 4-5 times with TBS-T for 1 hour. The Enhanced Chemiluminescence Detection System (Thermo Fischer) was used to visualize the immunostaining using ChemiDoc™ XRS+ System with Image Lab™ Software (Bio-Rad).

For the rodent studies, rat cerebrospinal fluid was extracted from 4-5 month old rats during stereotaxic surgery by direct puncture method to the cisterna magna at the base of the skull and aspirated using a 27G butterfly needle and a 1 ml syringe. The CSF was kept at −80° C. until needed for the Western blot. In humans, a lumbar puncture is used to obtain cerebrospinal fluid.

### Example 1—Rat Studies

The inventors have developed a new model for AS consisting of the deletion of the entire UBE3A gene. Using CRISPR/Cas 9 technology, a Ube3A-deficient rat was developed to have a Del 90457 bp/ins 8 bp (Termed Ube3A457). This model was used for the experiments performed herein and was found to allow for extraction of 100-200 ul of cerebrospinal fluid (CSF) and testing as to whether the UBE3A protein is present.

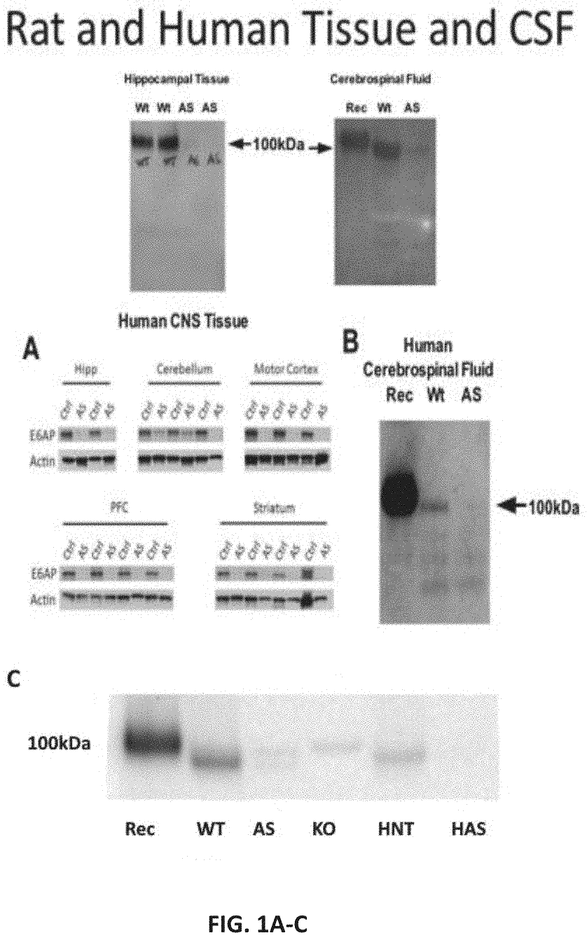

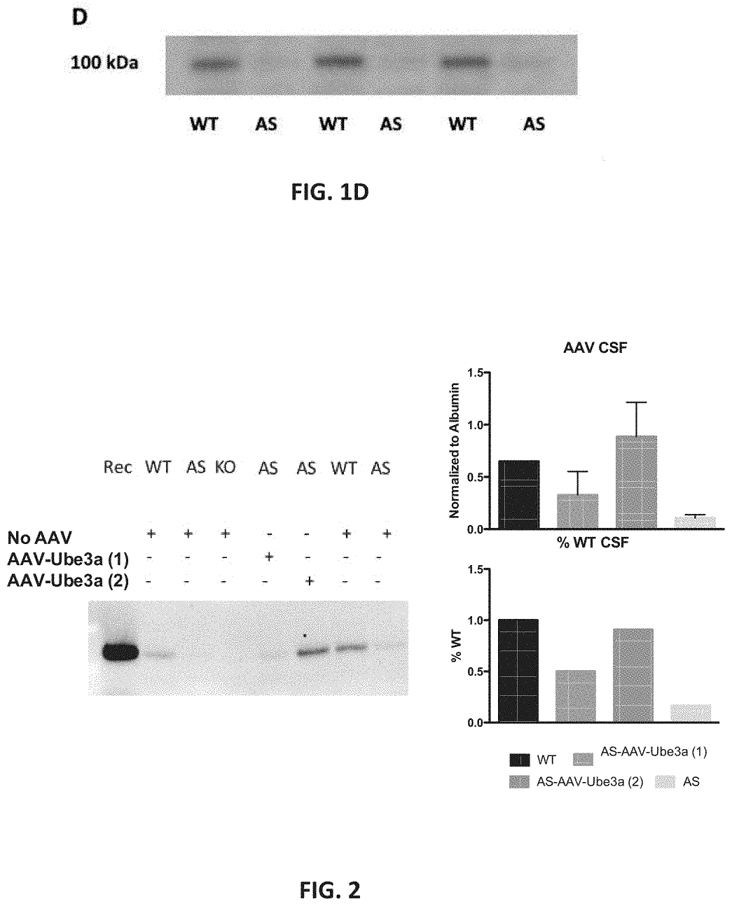

As noted previously, the absence of UBE3A can be indicative of AS. FIG. 1 illustrates an analysis of both human and rat brain tissue as well as CSF. As shown in the images, a lack of the maternal UBE3A allele results in a marked reduction of UBE3A protein in both hippocampal tissue as well as CSF. This reduction of UBE3A protein in the CSF is indicative that the CSF may be used to assist in a biochemical diagnosis of AS.

FIG. 2 further supports the use of CSF for biochemical diagnosis of AS. As shown in the figure, AAV-UBE3A mediated expression in the AS rat shows increased UBE3A in the CSF 5 weeks after viral transfection, indicating that UBE3A in the CSF is derived from neuronal expression and that exogenous gene therapy is sufficient to detect UBE3A in the CSF.

In view of these results and to allow for a quantitative biochemical diagnosis of AS, the inventors developed an enzymatic assay using a substrate for the UBE3A protein. UBE3A ubiquitinates any specific substrate, including the S5a protein. While S5a is used as the substrate in the assays of the examples, other substrates act in the same manner and can be used as an alternative to S5a. Examples of substrates that may be used include, but are not limited to, S5a, Sox9, Rad23, p53, MCMI, p2′7, promyelocytic leukemia tumor suppressor (PML), amplified in breast cancer 1 (AIB1), HHR23A, a-Synuclein, C/EBPa and itself, UBE3A. Analysis of the time course of the substrate ubiquitination allows a quantification of the activity of UBE3A from either human, mouse or rat.

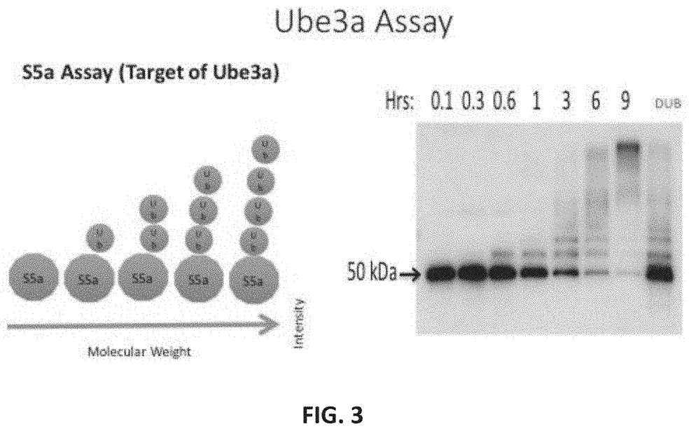

As shown in FIG. 3, CSF from a rat was incubated with recombinant S5a and stopped at specific time points. Analysis of the reduction of the 50 kDa S5a protein as it is ubiquinated and changes its molecular weight is performed. Deubiquitinating Enzymes (DUB) are proteases that reversely modify proteins by removing ubiquitin or ubiquitin-like molecules or remodeling ub-chains on target proteins. DUBs can be used in activity assays. In the instant case, the inventors used a DUB 9-hour assay in which UBE3A protein from CSF incubated with deubiquitinating enzymes allows for direct visualization of deubiquitinating enzyme activity and shows the increase in molecular weight is due to ubiquination.

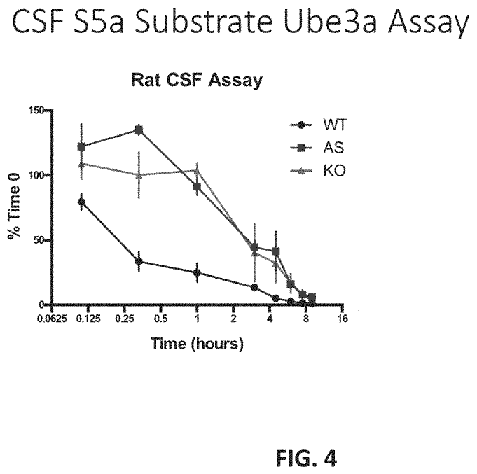

FIG. 4 illustrates results from a CSF UBE3A assay from CSF obtained from wild-type, UBE3A maternal deficient Rats (AS) and UBE3A knock out rats. Reduction over time of S5a substrate reveals that other ubiquitin ligases are present in Rat CSF that are not UBE3A, but the differential activity in the AS rat is due to UBE3A exclusively.

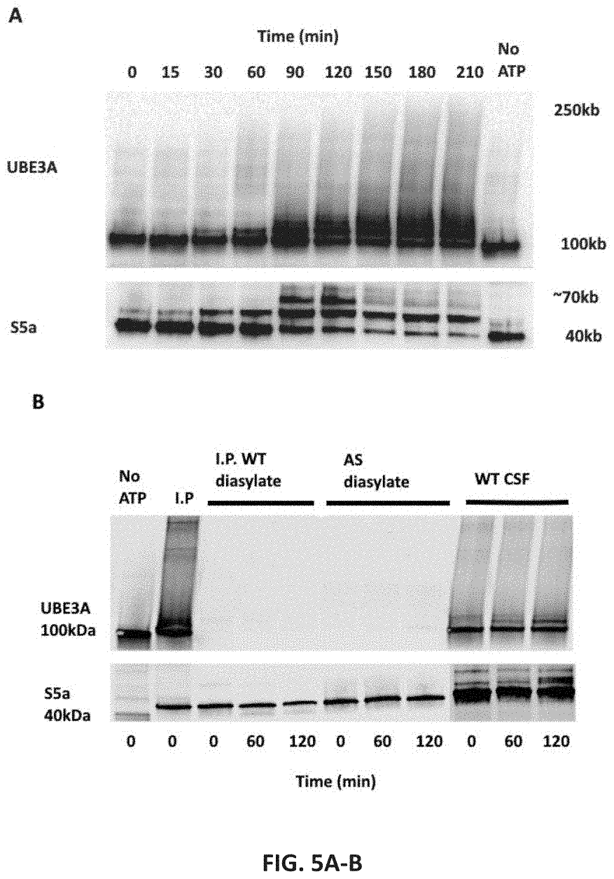

The absence of UBE3A protein the AS rat suggested not only a unique biomarker that can be easily assessed, but also that UBE3A has an extracellular function in the brain. The inventors conducted experiments to determine whether UBE3A protein is present in the extracellular space within the brain. Centrifuge tubes containing 10× reaction buffer, 10×Mg²⁺-ATP, 110×E2 enzyme (UBE2D3), diasylate, and 10× ubiquitin were placed in an incubator at 37° C. for the entirety of the assay. At each time point collection, 30 from each reaction tube were added to a tube with 5× loading buffer and 1 ul of 1M DTT, terminating the reaction. Samples were then flash frozen on dry ice and stored at −80° C. until Western blot analysis. As shown in FIG. 5, the UBE3A maintains its catalytic activity within the extracellular space.

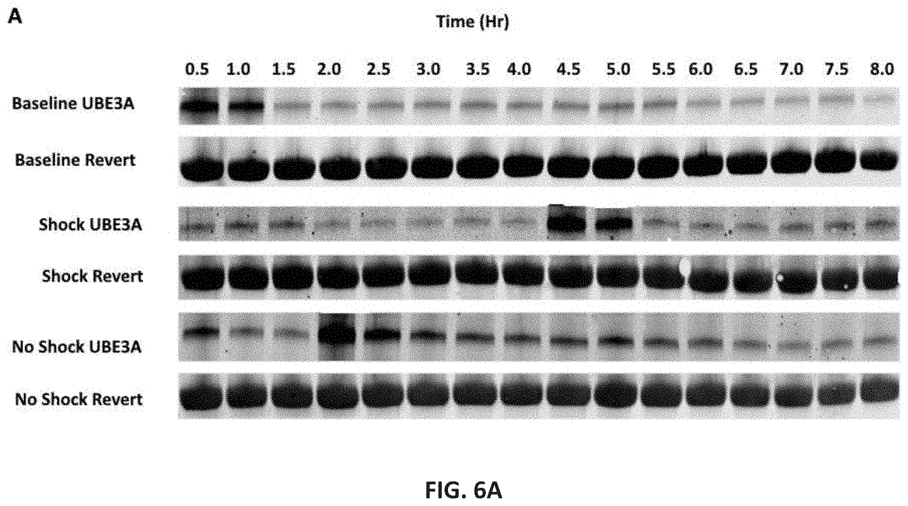

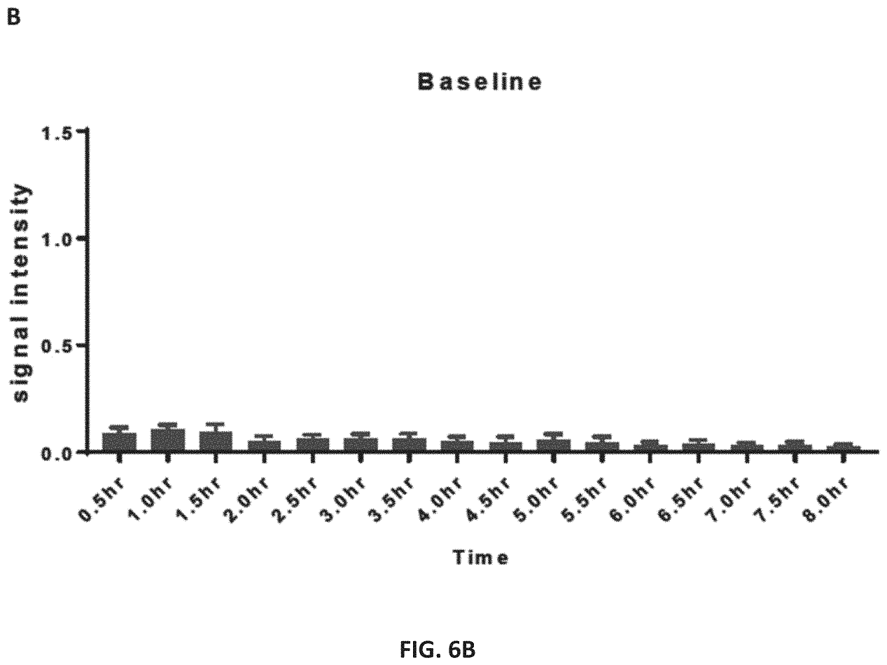

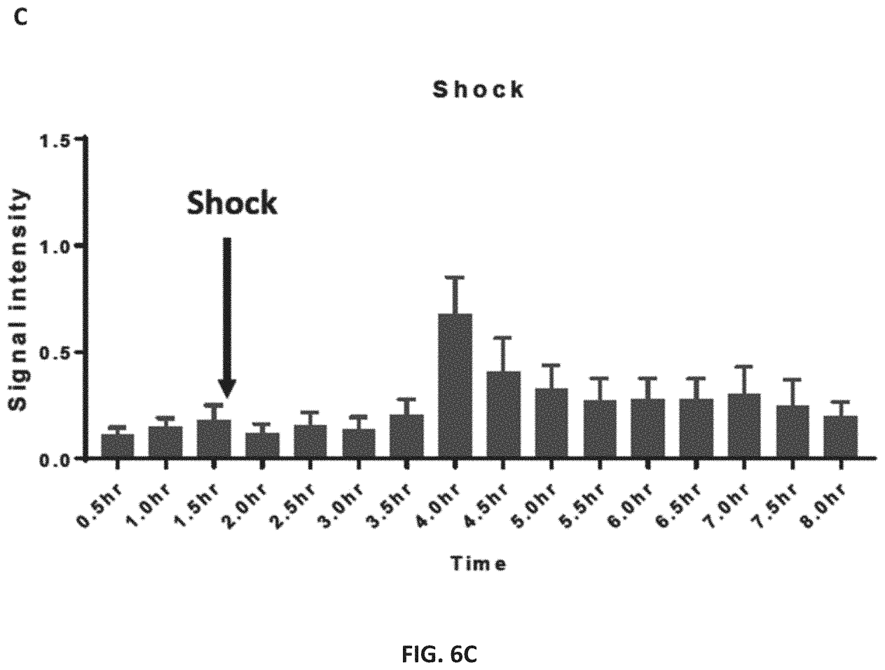

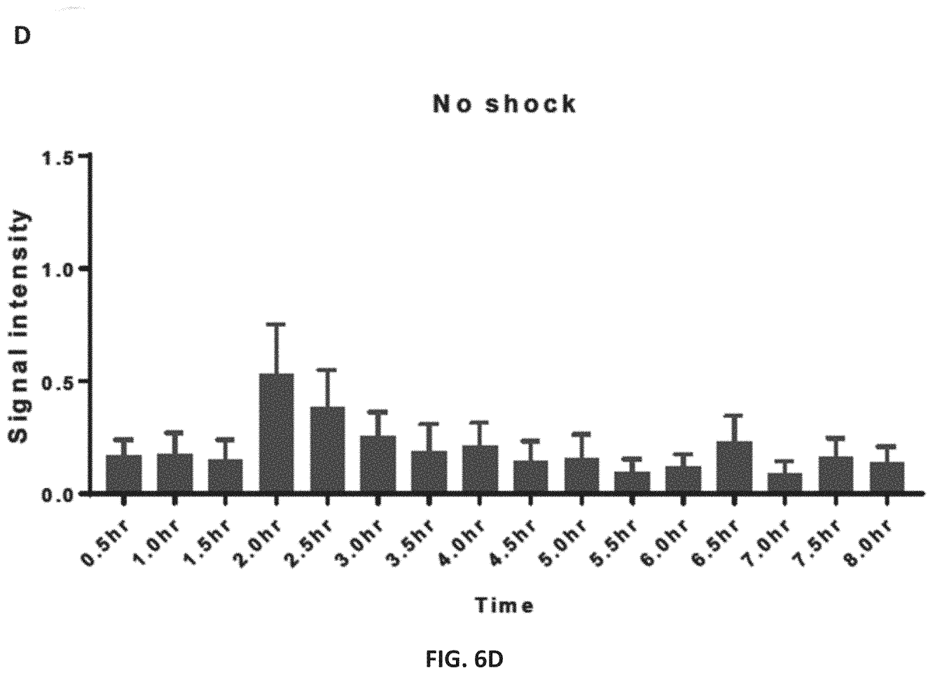

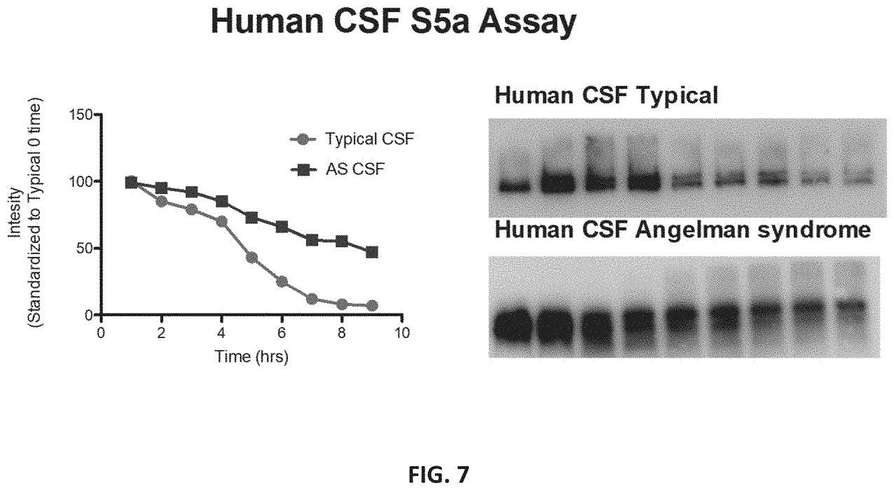

Hippocampal microdialysis was performed on rats to determine UBE3A presence in the extracellular space as a result of fear conditioning. Briefly, 4-5 month old rats were habituated for a week prior to left hippocampal cannula insertion conducted under carprofen (10 mg/kg) and isoflurane with insertion 5.6 mm post bregma, +5.0 lateral, and 3.0 ventral from the dura. After 48 hours, the rats were placed in a universal case/probe. 2 hours later a baseline collection was performed. The rats were placed in a fear conditioning chamber after 1.5 hours and remained in the fear conditioning chamber for 5 minutes before being placed back in the universal cage. After 8 hours, the rats were sacrificed. The results demonstrated that levels of extracellular UBE3A changes during active learning which implies an important function in synaptic plasticity. Specifically, extracellular UBE3A undergoes activity dependent regulation following fear conditioning. (FIG. 6) Changes in mouse UBE3A expression has also been shown to occur with learning in mice. Given the results, further investigation into the functional role of UBE3A in learning and memory will be conducted.

It was discovered that rat CSF from an AS rat has nominal UBE3A protein compared to a wild type rat as measured qualitatively with Western blot analysis. The same enzymatic assay and Western blot analysis were used on human CSF from a de-identified 11-year-old female AS patient. As discussed in Example 2 below, the same results were shown in which UBE3A enzymatic activity was found to be nearly absent in the CSF collected from both the AS rat and the Human AS. (FIG. 7)

### Example 2—Human Study

For the human study, the assay was performed on CSF obtained from the 11 yo AS patient only as CSF samples from AS patients are relatively rare to come by outside of a clinical trial. To the inventors' knowledge, they are the only laboratory that has had access to CSF from an AS patient as these types of samples are not available through the NIH, which is indicative of their rarity. Only one neurotypical CSF control was included for the assay, however CSF from additional control subjects can be obtained.

For the assay, cerebrospinal fluid (CSF) was collected from an 11-year-old patient diagnosed with AS as described in the protocols above. Briefly, the CSF was first concentrated and then an amount of CSF was added to a reaction tube with 10×E2 enzyme, Mg²⁺-ATP solution, and His6-55a substrate protein. 10× ubiquitin was added to the reaction tube. The reaction tubes were then placed in an incubator at 37° C. for the entirety of the assay. At each time point collection, 3 μl from each reaction tube were added to a tube with 5× loading buffer and 1 ul of 1M DTT, terminating the reaction. Western blots were then performed for each timepoint to measure the amount of ubiquination of the S5a substrate by UBE3A.

As shown in FIG. 5, when comparing the result to that of a normal, control subject, it was found that the AS patient exhibited nominal UBE3A enzymatic activity as compared to the control. As shown in the figure, S5a ubiquitination over time was examined for human CSF from a normal control subject (typical) compared to CSF from an AS source. Human typical CSF has significantly more ability to ubiquitinate S5a compared to CSF from AS which is due to the presence of an increased amount of UBE3A in the normal subject.

### Example 3—Prophetic Use as a Biomarker to Determine Treatment Efficacy

A 12-year-old patient diagnosed with AS is treated with AAV-mediated gene therapy in which a therapeutically effective amount of UBE3A vector is injected bilaterally into the left and right hippocampal hemispheres of the brain. CSF is collected prior to administration of treatment and at defined time periods after treatment by lumbar puncture. The CSF sample collected prior to administration of treatment is used as the reference sample. The amount of UBE3A in each CSF sample is quantified and compared to the reference sample as described. Briefly, the CSF is first concentrated and then an amount of CSF is added to a reaction tube with 10×E2 enzyme, Mg²⁺-ATP solution, and His6-S5a substrate protein. 10× ubiquitin is added to the reaction tube. The reaction tubes are then placed in an incubator at 37° C. for the entirety of the assay. At each time point collection, 30 from each reaction tube are added to a tube with 5× loading buffer and 1 ul of 1M DTT, terminating the reaction. Western blots are then performed for each timepoint to measure the amount of ubiquitination of the S5a substrate by UBE3A. An increase in UBE3A in the patient's CSF sample, as measured by an increase in ubiquitination of the S5a substrate, is indicative of efficacious treatment. An amount of UBE3A equal to or less than the reference sample amount, as measured by an equal or decrease in ubiquitination of the S5a substrate, is indicative of inefficacious treatment.

### Example 4—Prophetic Use to Diagnose AS

A 5-year-old patient presents with developmental delays that started around the age of 12 months. The patient presents with absent speech, seizures, hypotonia, ataxia and microcephaly. The child moves with a jerky, puppet like gait and displays an unusually happy demeanor that is accompanied by laughing spells. The child has dysmorphic facial features characterized by a prominent chin, an unusually wide smile, and deep-set eyes.

A lumbar puncture is performed on the child to obtain a sample of CSF for biochemical diagnosis of AS. A CSF sample from a normal individual is used as a control sample. The CSF of both the patient and the normal individual are first concentrated and then an amount of the respective CSF is added to a reaction tube with 10×E2 enzyme, Mg²⁺-ATP solution, and His6-S5a substrate protein. 10× ubiquitin is added to the reaction tube. The reaction tubes are then placed in an incubator at 37° C. for the entirety of the assay. At each time point collection, 3 μl from each reaction tube are added to a tube with 5× loading buffer and 1 ul of 1M DTT, terminating the reaction. Western blots are then performed for each timepoint to measure the amount of ubiquitination of the S5a substrate by UBE3A. The Western blots of the patient are compared to that of the normal control sample. It is found that the patient's sample exhibited only nominal UBE3A enzymatic activity as compared to the control sample thus allowing for a biochemical confirmation of an AS diagnosis for the patient.

The advantages set forth above, and those made apparent from the foregoing description, are efficiently attained. Since certain changes may be made in the above construction without departing from the scope of the invention, it is intended that all matters contained in the foregoing description or shown in the accompanying drawings shall be interpreted as illustrative and not in a limiting sense.

All referenced publications are incorporated herein by reference in their entirety. Furthermore, where a definition or use of a term in a reference, which is incorporated by reference herein, is inconsistent or contrary to the definition of that term provided herein, the definition of that term provided herein applies and the definition of that term in the reference does not apply.

While certain aspects of conventional technologies have been discussed to facilitate disclosure of the invention, Applicants in no way disclaim these technical aspects, and it is contemplated that the claimed invention may encompass one or more of the conventional technical aspects discussed herein.

The present invention may address one or more of the problems and deficiencies of the prior art discussed above. However, it is contemplated that the invention may prove useful in addressing other problems and deficiencies in a number of technical areas. Therefore, the claimed invention should not necessarily be construed as limited to addressing any of the particular problems or deficiencies discussed herein.

In this specification, where a document, act or item of knowledge is referred to or discussed, this reference or discussion is not an admission that the document, act or item of knowledge or any combination thereof was at the priority date, publicly available, known to the public, part of common general knowledge, or otherwise constitutes prior art under the applicable statutory provisions; or is known to be relevant to an attempt to solve any problem with which this specification is concerned.

It is also to be understood that the following claims are intended to cover all of the generic and specific features of the invention herein described, and all statements of the scope of the invention which, as a matter of language, might be said to fall there between. Now that the invention has been described,

What is claimed is:

1. A method of diagnosing and treating a neurodegenerative disease characterized by UBE3A deficiency in a patient comprising:
extracting a sample of cerebrospinal fluid from the patient;
combining the sample of cerebrospinal fluid with a substrate protein and ubiquitin in a reaction tube;
incubating the reaction tube;
terminating reaction of the contents at set time points;
quantifying UBE3A enzymatic activity in the patient sample;
comparing the UBE3A enzymatic activity of the patient sample to a control sample; and
administering treatment for the neurodegenerative disease if an absence or decrease of UBE3A enzymatic activity in the patient sample as compared to the control sample is found.

2. The method of claim 1, wherein the neurodegenerative disease is selected from the group consisting of Angelman's Syndrome, Huntington's disease, Alzheimer's disease, Parkinson's disease, amyotrophic lateral sclerosis, autistic spectrum disorders, epilepsy, multiple sclerosis, Prader-Willi syndrome, Fragile X syndrome, Rett syndrome and Pick's Disease.

3. The method of claim 1, wherein the neurodegenerative disease is Angelman syndrome.

4. The method of claim 1, wherein the substrate protein is selected from the group consisting of S5a, Sox9, Rad23, p53, MCMI, p2′7, promyelocytic leukemia tumor suppressor (PML), amplified in breast cancer 1 (AIB1), HHR23A, a-Synuclein, C/EBPa and UBE3A.

5. The method of claim 1, wherein the substrate protein is S5a.

6. The method of claim 1, wherein the UBE3A enzymatic activity is quantified by measuring the amount of ubiquitination of the substrate protein.

7. The method of claim 6, wherein a Western blot is used to measure the amount of ubiquitination of the substrate protein.

8. The method of claim 1, wherein the treatment is selected from the group consisting of activation of the paternal allele, AAV-mediated gene therapy and protein replacement therapy.

9. A method of determining efficacy of treatment of a neurodegenerative disease characterized by UBE3A deficiency in a patient comprising:
collecting a reference sample of cerebrospinal fluid from the patient prior to administering treatment;
administering a treatment to the patient having Angelman Syndrome;
collecting at least one sample of cerebrospinal fluid from the patient at least one time period after treatment;
incubating the reference sample with a substrate protein and ubiquitin in a first reaction tube;
incubating the at least one sample collected at the at least one time period after the administration of the treatment with the substrate protein and the ubiquitin in a second reaction tube;
terminating reaction of the contents of each of the reaction tubes at set time points;
quantifying UBE3A enzymatic activity in both the reference sample and the at least one sample collected at the at least one time period after the administration of the treatment; and
comparing the UBE3A enzymatic activity in the at least one sample collected at the at least one time period after the administration of the treatment to the reference sample;
wherein an increase in the amount of UBE3A enzymatic activity in the at least one sample collected at the at least one time period after the administration of the treatment as compared to the reference sample indicates efficacious treatment.

10. The method of claim 9, wherein the neurodegenerative disease is selected from the group consisting of Angelman's Syndrome, Huntington's disease, Alzheimer's disease, Parkinson's disease, amyotrophic lateral sclerosis, autistic spectrum disorders, epilepsy, multiple sclerosis, Prader-Willi syndrome, Fragile X syndrome, Rett syndrome and Pick's Disease.

11. The method of claim 9, wherein the neurodegenerative disease is Angelman syndrome.

12. The method of claim 9, wherein the substrate protein is selected from the group consisting of S5a, Sox9, Rad23, p53, MCMI, p2′7, promyelocytic leukemia tumor suppressor (PML), amplified in breast cancer 1 (AIB1), HHR23A, a-Synuclein, C/EBPa and UBE3A.

13. The method of claim 9, wherein the substrate protein is S5a.

14. The method of claim 9, wherein the UBE3A enzymatic activity is quantified by measuring the amount of ubiquitination of the substrate protein.

15. The method of claim 14, wherein a Western blot is used to measure the amount of ubiquitination of the substrate protein.

16. The method of claim 9, wherein the treatment is selected from the group consisting of activation of the paternal allele, AAV-mediated gene therapy and protein replacement therapy.

17. A kit for diagnosing Angelman's Syndrome comprising:
a substrate protein wherein the substrate protein is selected from the group consisting of S5a, Sox9, Rad23, p53, MCMI, p2′7, promyelocytic leukemia tumor suppressor (PML), amplified in breast cancer 1 (AIB1), HHR23A, a-Synuclein, C/EBPa and UBE3A;
an enzyme solution;
an adenosine triphosphate (ATP) solution;
a ubiquitin solution;
a redox reagent; and
printed instructions for use of the kit in diagnosing Angelman's syndrome using cerebrospinal fluid from a subject;
wherein all components of the kit are contained in separate containers.

18. The kit of claim 17, wherein the redox reagent is dithiothreitol (DTT).
